(19) United States
(12) Patent Application Publication
(10) Pub. No.: US 2024/0329021 A1
(43) Pub. Date: Oct. 3, 2024

(54) DYNAMIC MEASUREMENT OF AQUEOUS AMINE CARBON DIOXIDE ABSORPTION

(71) Applicant: INTERNATIONAL BUSINESS MACHINES CORPORATION, ARMONK, NY (US)

(72) Inventors: Theodore G. van Kessel, Millbrook, NY (US); Stacey Gifford, Ridgefield, CT (US); Benjamin Hardy Wunsch, Mt. Kisco, NY (US)

(21) Appl. No.: 18/190,228

(22) Filed: Mar. 27, 2023

(51) Int. Cl. G01N 33/00; B01L 3/00; B01L 7/00; G01N 1/42; G01N 1/44; G01N 11/00

(52) U.S. Cl. CPC G01N 33/0027; B01L 3/502723; B01L 7/52; G01N 1/42; G01N 1/44; G01N 11/00; B01L 2200/16; B01L 2300/18; B01L 2400/0622

(57) ABSTRACT

A system may comprise a heat spreader with a sample holder, an amine solution in the sample holder; a temperature sensor configured to measure a temperature of the amine solution; a heating and cooling device in the spreader, a gas flow valve connected to a gas inlet tube, wherein an end of the gas inlet tube sits below a surface level of the amine solution; and a CO2 (carbon dioxide) sensor connected to a gas outlet tube.

### BACKGROUND

Aspects of the present disclosure relate to the dynamic measurement of aqueous amine carbon dioxide absorption kinetics parameters.

Carbon capture and sequestration is a central focus of efforts to reduce the rate of climate change due to greenhouse gas accumulation in the atmosphere.

Carbon capture relies on the ability to separate carbon dioxide (CO2) gas from other gasses at typically low concentrations (15% to 30%) and purify the gas to 90% or better.

One method of carbon capture involves the use of aqueous amine solutions to absorb CO2 from flue gas streams at low temperature and concentration. The solution is then heated, and the absorbed CO2 is released at high concentration suitable for subsequent liquefaction or downstream use.

### BRIEF SUMMARY

The present disclosure provides a system and methods of dynamic measurement of aqueous amine carbon dioxide absorption kinetics parameters. In some embodiments, the system includes a heat spreader with a sample holder; an amine solution in the sample holder; a thermocouple configured to measure a temperature of the amine solution; a heating and cooling device in the spreader; a gas flow valve connected to a gas inlet tube, wherein an end of the gas inlet tube sits below a surface level of the amine solution; and a CO2 (carbon dioxide) sensor connected to a gas outlet tube.

Some embodiments of a method comprises placing an amine solution in a sample holder; placing the sample holder in a receptacle in a heat spreader; bringing a temperature of the amine solution to a desired temperature value; valving a specified gas volume with a CO2 concentration to the amine solution for a period of time sufficient to saturate the amine while monitoring a CO2 exhaust concentration using a CO2 sensor; thermal cycling the amine solution by alternately heating and cooling the amine solution for fixed time periods and a number of cycles; and monitoring the CO2 exhaust concentration.

Some embodiments of a method comprises determining, based on a result of a CO2 (carbon dioxide) absorption test, a capacity of an amine solution and an initial concentration of absorbed CO2; determining first order reaction kinetics parameters of the amine solution; determining a change in amine concentration verses time; and modifying a manufacturing process based on the first order reaction kinetics parameters of the amine solution and the change in amine concentration verses time.

### BRIEF DESCRIPTION OF THE DRAWINGS

FIG. 1 depicts a system for testing the CO2 absorption parameters for an amine solution, according to various embodiments of the present invention.

FIG. 2 illustrates an example thermal cycling sub system, according to various embodiments of the present invention.

FIG. 3 depicts a graph of data taken for aqueous amine carbon dioxide absorption kinetics parameters taken during presoak and thermal cycling, according to various embodiments of the present invention.

FIG. 4 depicts an example method for dynamic measurement of aqueous amine carbon dioxide absorption kinetics parameters, according to various embodiments of the present invention.

FIG. 5 depicts an example method for predicting the behavior of the amine solution under varying conditions, according to various embodiments of the present invention.

FIG. 6 depicts a graph illustrating the results of a test determining the cumulative CO2 absorbed during presoak, according to various embodiments of the present invention.

FIG. 7 depicts a graph illustrating the estimated forward and reverse activation energies for CO2 absorption in various aqueous amine solutions, according to various embodiments of the present invention.

FIG. 8 depicts a graph illustrating the estimated forward and reverse pre-exponential factors (rate of the process) for CO2 absorption in various aqueous amine solutions, according to various embodiments of the present invention.

FIG. 9 depicts a computer system according to various embodiments of the present invention.

### DETAILED DESCRIPTION

Aspects of the present disclosure relate to dynamic measurement of aqueous amine carbon dioxide absorption kinetics parameters. While the present disclosure is not necessarily limited to such applications, various aspects of the disclosure may be appreciated through a discussion of various examples using this context.

Carbon capture and sequestration is a central focus of efforts to reduce the rate of climate change due to greenhouse gas accumulation in the atmosphere. Carbon capture relies on the ability to separate carbon dioxide (CO2) gas from other gasses at relatively lower concentrations (e.g., 15% to 30%, 10% to 40%, etc.) and purify the gas to 90% or higher. In some instances, one method of carbon capture involves the use of aqueous amine solutions (referred to herein as amine solution, amine, or sample) to absorb CO2 from flue gas streams at low temperature and concentration. The solution is then heated, and the absorbed CO2 is released at high concentration suitable for subsequent liquefaction or downstream use. The solution is then cooled and the cycle repeated.

The temperature swing process referred to above expends significant amounts of energy to heat and cool the aqueous amine solution to capture and release the gas. There are many types of amines and each has different capacities and rates of reaction. These parameters impact the energy required to carry out the temperature swing process and the associated throughput of CO2. Therefore, in some embodiments, a method is presented to rapidly measure and analyze small quantities of an aqueous amine solution and compute the corresponding CO2 capture capacity and reaction kinetic parameters, thereby allowing CO2 processes to be efficiently and precisely tailored for an application.

In some embodiments, the system proposed rapidly cycles the sample amine solution through a user defined temperature trajectory while measuring absorbed and desorbed CO2 from a known gas stream (e.g., the composition of the gas stream is known) thereby providing the dynamic response data for the sample amine solution that allows the analysis and determination of the reaction kinetic parameters.

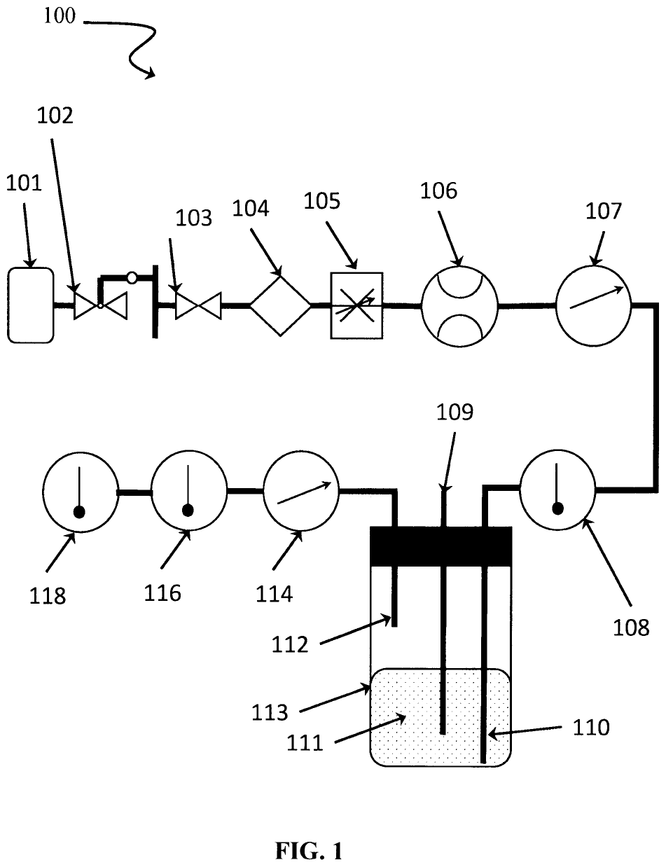

FIG. 1 depicts a system 100 for testing the CO2 absorption parameters for an amine solution. In exemplary FIG. 1, a gas mixture containing a known percentage of CO2 and other gases (for example, 10% CO₂ in N2) is valved from tank 101 to a mass flow controller 105 which is set to a specific flow rate. For example, the flow rate may be 15 sccm (Standard Cubic Centimeters per Minute), other flow rates are possible and considered by this disclosure. In some instances, time variable flow profiles are also possible. For example, the flow rate may range from 10 sccm to 20 sccm. In some embodiments, multiple gas tanks and regulators are used to allow gas mixtures to be created. In this case the exit flows from each mass flow controller are joined to the single line prior to a flow meter 106.

In some embodiments, the gas may flow through a pressure regulator 102, a valve 103, and a filter 104. In some instances, the pressure regulator 102 is a device that is used on a gas line to regulate the pressure of the gas being delivered to an appliance or system.

In some instances, the tank 101 may be at a high pressure, but the system may require a lower pressure to operate safely and efficiently. In some instances pressure regulator 102 reduces the pressure of the gas coming from the gas line to a lower, more manageable level before it enters the rest of the system 100.

In some embodiments, the gas stream flows through flow meter 106, pressure meter 107, and temperature sensor 108 to a test cell 113 containing an amine solution 111 to be tested. In some instances, temperature sensors can be based on a variety of technologies, including thermistors, RTDs (resistance temperature detectors), and thermocouples. For example, the amine solution size may be 100 to 200 uL (microliters). In some embodiments, a thermocouple sensor 109 is placed in test cell 113 to monitor the temperature of the solution during a test. In some embodiments, the gas stream is bubbled through amine solution 111 through a thin gas inlet tube 110. As depicted, an end of the gas inlet tube 110 may sit below the surface of the amine solution 111. For example, the end of the gas inlet tube 110 may be situated below the surface of the amine solution 111 or a bubbling device may be attached to the end of gas inlet tube 110 and the bubbling device may be situated below the surface of amine solution 111.

In some instances, a flow meter for gas is a device that measures the flow rate of gas moving through a pipeline or system. The flow rate is typically measured in volume per unit time, such as cubic feet per hour (CFH), standard cubic centimeters per minute (SCCM), or cubic meters per hour (CMH). In some instances, a pressure meter for gas is a device used to measure the pressure of gas within a pipeline or system. Gas pressure meters can be mechanical or electronic and are designed to measure the pressure in one or more different units, such as pounds per square inch (PSI) or bar.

In some instances, valve 103 may be used to shut off or turn on gas flow completely from tank 101. In some instances, filter 104 may remove particulates or other contaminants from the gas coming from tank 101.

In some embodiments, the gas then flows through a thin gas outlet tube 112 downstream to a CO2 sensor 114, a relative humidity 116 sensor, and a temperature sensor 118. For example, the CO2 sensor 114 types include but are not limited to Nondispersive Infrared Sensor (NDIR), thermal, and photoacoustic sensors.

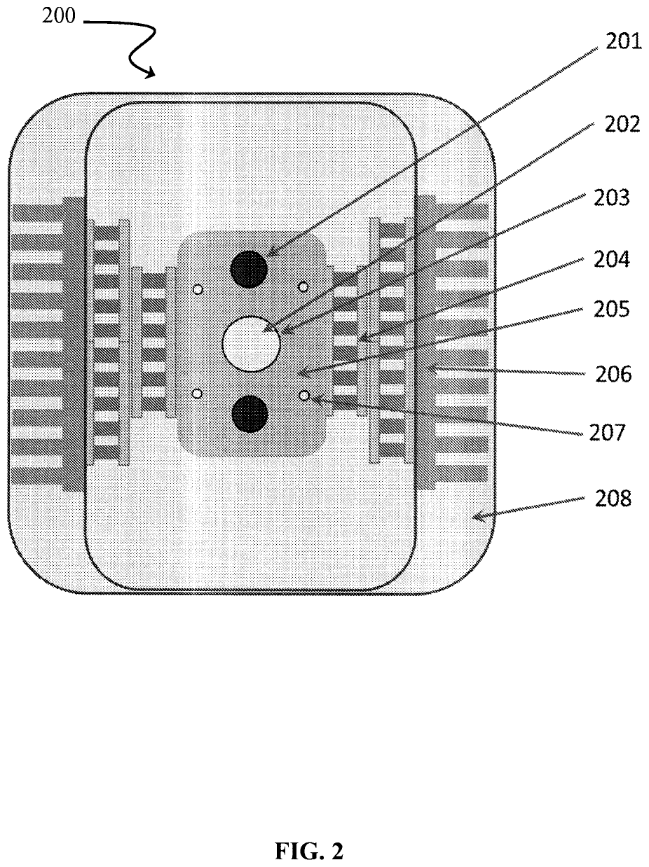

FIG. 2 illustrates an example thermal cycling sub system 200. In some embodiments, sub-system 200 is designed to fit around sample cell 113. Although the sample cell is depicted as a cylinder, other shapes are contemplated.

In some embodiments, during operation, the glass sample cell 113 is placed in a cylindrical receptacle 202 in a heat spreader 205 with an appropriate thermal interface grease. In practice, vacuum grease, or other suitable material (not shown) may be used to assure good thermal contact between the sample cell and the heat spreader 205. For example, the sample size may be 100 uL to 200 uL, but other sample sizes may be used.

In some embodiments, during operation, cartridge heaters 201 and thermo-electric (Peltier) modules 204 are used to heat and cool the amine solution (e.g., the sample) in sample holder 202 through the heat spreader 205 (e.g., a copper heat spreader or a thermal spreader). Waste heat (e.g., heat removed during the cooling phase) is removed or dissipated from the system using fan sinks 206 and transferred (i.e., dissipated outside the system) by cooling duct 208. In embodiments without the cooling duct 208, the fan sinks 206 simply transfer the heated air to the ambient environment. In some embodiments, the thermal cycling is partially actuated by a thermo-electric module and partially actuated by another heating system such as cartridge heaters 201. In some embodiments, the thermal cycling will have alternating cooling and heating phases.

In some instances, an ultrasonic liner 203 lining sample holder 202 may be used to produce ultrasonic vibration. For example, PVDF (polyvinylidene fluoride) liner is a material used in ultrasound imaging applications as a thin film or coating on the surface of transducers. The primary function of the PVDF liner is to act as a piezoelectric material, which generates and receives ultrasound waves. Ultrasound waves are generated by applying an electrical voltage to the PVDF liner, which causes it to vibrate and produce sound waves. The sound waves are transmitted through the sample, and when they bounce back, the PVDF liner receives the waves and converts them into electrical signals. These electrical signals are then processed to observe the sample under test with respect to whether it is changing behavior (e.g., change in state, viscosity, density, flow rate, composition, etc.). In alternate embodiments, the ultrasonic liner is used to agitate the sample under test.

In some instances, the thermo-electric module 204, for example a Peltier module, is an electronic component that can be used to generate a temperature difference between its two sides when a current is passed through it. It works on the principle of the Peltier effect, which is the phenomenon that occurs when an electrical current is passed through a junction of two different metals or semiconductors, causing heat to be transferred from one side to the other. For example, a Peltier module is typically made up of two thin ceramic plates, each of which has a number of semiconductor elements sandwiched between them. When an electric current is passed through the module, heat is transferred from one ceramic plate to the other, creating a temperature difference between the two sides. This effect can be used for both heating and cooling applications, depending on the direction of the current flow.

In some instances, gas supply heat exchange duct 207 is a component of a heating system that is used to pre-heat the input gases.

In some embodiments, a computer (e.g., computing environment 900) is used to control the power to the system elements and to read the sensors.

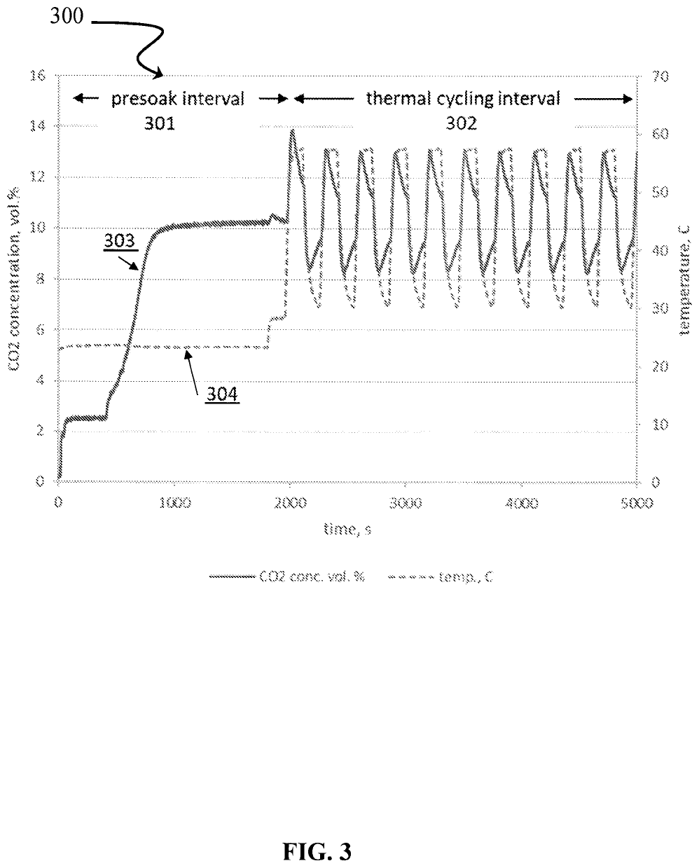

FIG. 3 depicts a graph 300 of data taken for aqueous amine carbon dioxide absorption kinetics parameters taken during presoak and thermal cycling using the system depicted in FIG. 1 and FIG. 2, the flow of CO2 was constant and assured using mass flow controller 105 (although it may be varied as described above). In some instances, presoaking is leaving the amine at a constant temperature to saturate the amine with CO2 for a the given temperature, given CO2 concentration of the gas, and given gas flow rate. A flow value of 15 SCCM was used. The inlet CO2 gas concentration was assured by precision mix in tank 101. In this example, there was a 10% volume concentration of CO2. FIG. 3 shows the CO2 output concentration during presoak time period 301 and thermal cycling periods 302. In this test, sensor data and system state variables were recorded at approximately one second intervals or less. The outlet concentration of CO2 is depicted with solid line 303 and the sample temperature is depicted with the dashed line 304. In some instances, graph demonstrates the ability of an amine solution to absorb and release CO2 with thermal cycling. For example, line 303n (CO2 concentration) fluctuates with line 304 (temperature) because the amine solution absorb or release CO2 depending on the temperature of the amine solution.

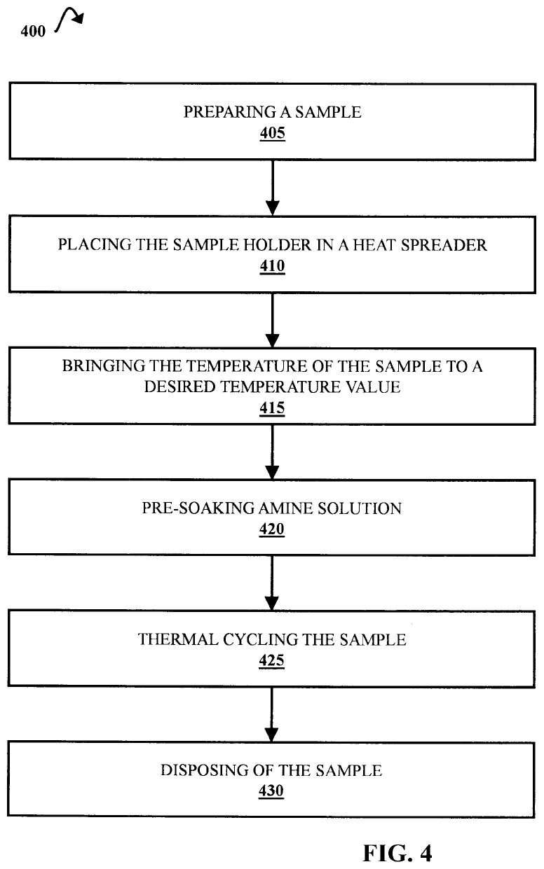

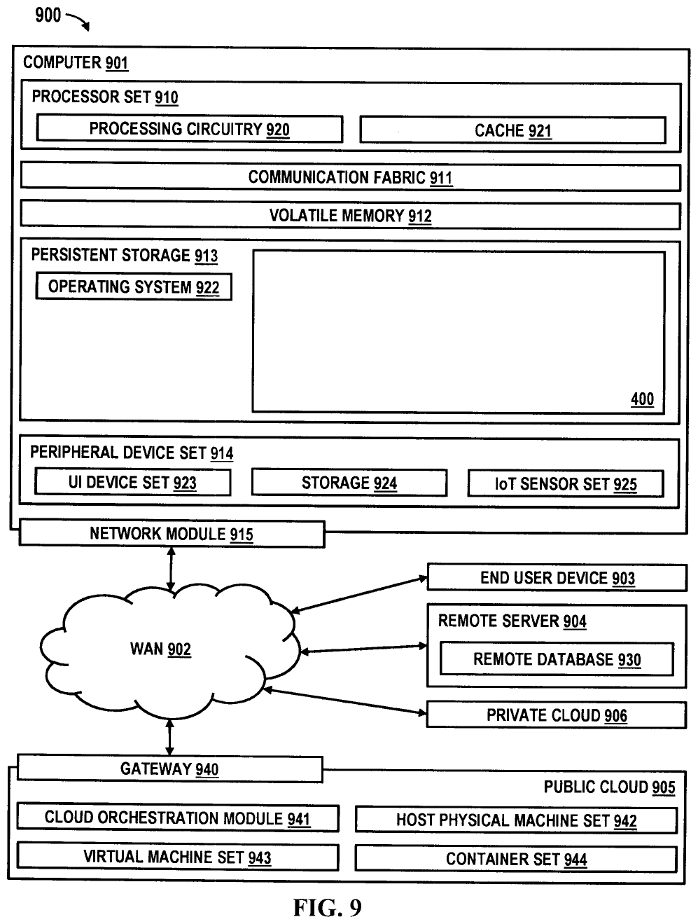

FIG. 4 depicts an example method 400 for dynamic measurement of aqueous amine carbon dioxide absorption kinetics parameters. Operations of method 400 may be enacted by one or more computer systems, such as the system described in FIG. 9 below.

Method 400 begins with operation 405 of preparing a sample by loading a quantity of amine solution in the glass sample holder.

Method 400 continues with operation 410 of placing the sample holder in the receptacle in a heat spreader (e.g., a copper spreader block or heat spreader 205).

Operation 415 includes bringing the temperature of the sample to a desired temperature value by appropriate heating or cooling using a thermocouple (e.g., thermocouple 109) as a reference.

Operation 420 includes valving a specified CO2 gas volume and concentration to the sample for a period of time sufficient to saturate the amine with CO2 while monitoring the CO2 exhaust concentration using CO2 sensor (e.g., sensor 114) to detect when saturation has been achieved. Herein, operation 420 may be referred to as a “pre-soak.” In some instances, pre-soaking saturation levels may depend on the concentration of CO2 for incoming gas, temperature of the amine, and humidity of the gas. In some embodiments, the amine solution may be saturated when the concentration of CO2 of gas leaving the amine solution has reached a constant value.

Method 400 continues with operation 425 of thermal cycling the sample by alternately heating and cooling the sample for pre-determined time periods and number of cycles while monitoring the CO2 exhaust concentration, temperature, pressure, and relative humidity. For example, see the results depicted in FIG. 3. In some instances, after reaching a presoaking, heating and cooling the amine may case the amine to respectively absorb and release CO2. Thus, CO2 may be extracted from an incoming gas by heating the amine solution and later be released for collection by cooling the amine solution.

Method 400 may end with operation 430 of disposing of the sample. In some embodiments, the sample may be cooled to room temperature before disposal.

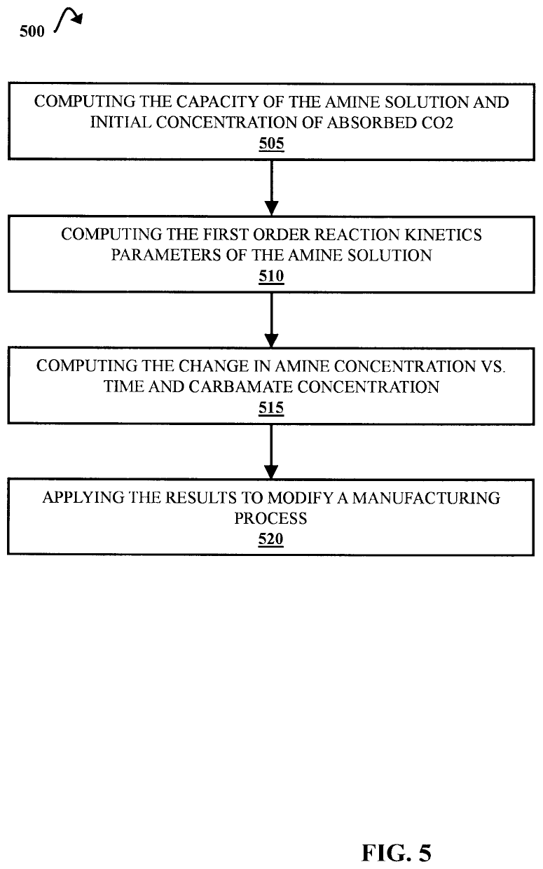

FIG. 5 depicts an example method 500 for predicting the behavior of the amine solution under varying conditions to improve use parameters and cost of operations. In some instances, method 500 may be predicted for actual use situations and plant models Operations of method 500 may be enacted by one or more computer systems such as the system described in FIG. 9 below.

Method 500 may begin with operation 505 of computing the capacity of the amine solution and initial concentration of absorbed CO2.

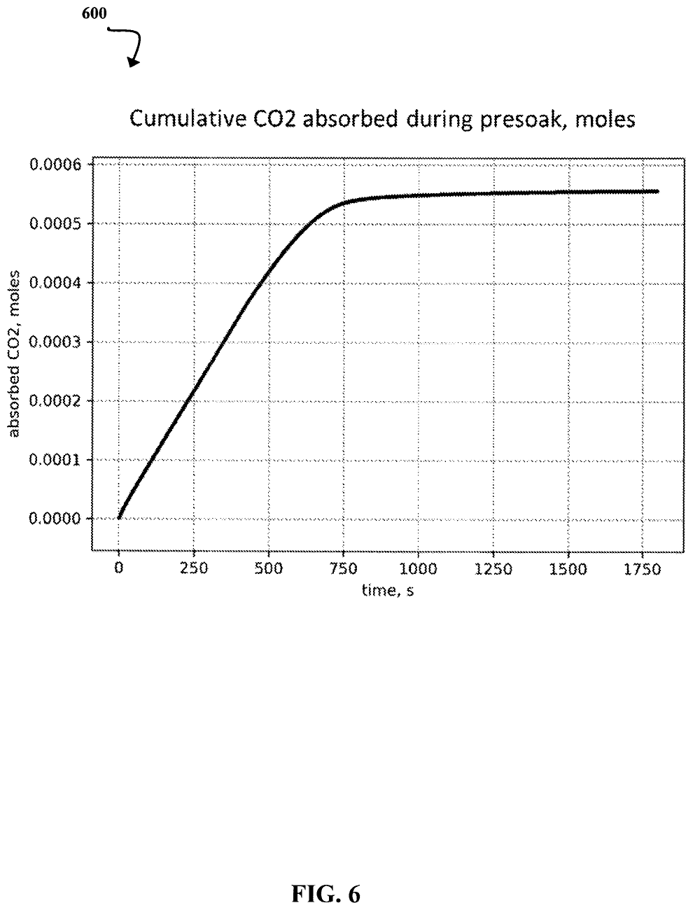

In some embodiments, given a known input flow volume CO2 and a concentration of a gas before and after passing through the amine solution; the net molar amount of CO2 absorbed in the sample can be computed by the difference of the 2 concentrations (before and after). Integrating this quantity over the presoak period results in the amount of CO2 absorbed in the sample. For example, FIG. 6 depicts graph 600 illustrating the results of a test determining the cumulative CO2 absorbed during presoak. In graph 600, the number of moles of amine present in the sample solution is computed from the known sample volume and the percentage of amine by weight.

In some embodiments, the capacity of the amine to absorb CO2 is computed by dividing the moles of CO2 absorbed divided by the moles of amine in the sample. The capacity of CO2 absorption can be compared in many cases with the known values for particular amines in the literature and serves as an initial condition for subsequent calculations.

Referring back to FIG. 5, the method 500 continues with operation 510 of computing the first order reaction kinetics parameters of the amine solution for the Arrhenius or Eyring form of reversable nth order reaction kinetics appropriate to the amine sample. Given the concentrations and temperatures shown above for the thermal cycling period 302, the system may be determined by computing the CO2 absorption of the sample versus time.

Method 500 may continue with operation 515 of computing the change in amine concentration vs. time and carbamate concentration. In some instances, since carbamate is formed in the forward reaction (absorption) and continuity, it is possible to compute the change in amine concentration vs time and the associated changes in carbamate concentration etc. In some instances, continuity refers to the principle of mass conservation, which states that the total mass of a closed system remains constant over time, barring any external inputs or outputs. The initial concentrations of carbamate and amine are known from operation 505.

In some instances, the Arrhenius equation and the Eyring equation are two different formulations used to describe the kinetics of reversible nth order reactions. Both the Arrhenius equation and the Eyring equation relate the rate constant (k) of a reaction to the temperature (T) and other relevant physical constants.

The Arrhenius equation is given by:

$$k = A * exp ( - Ea / RT )$$

where A is the pre-exponential factor, Ea is the activation energy for the reaction, R is the universal gas constant, and T is the temperature (e.g., in kelvin). This equation relates the (k) rate constant to the activation energy required to overcome the energy barrier for the reaction to occur. As the temperature increases, the rate constant increases due to the increased kinetic energy of the reactants, which allows more collisions to occur and thus more successful reactions. This equation is commonly used for simple reactions involving a single transition state.

The Eyring equation is given by:

$$k = ( k\_b * Th ) * exp ( - Δ G‡ / RT )$$

where kb is the Boltzmann constant, h is Planck's constant, ΔG‡ is the Gibbs free energy of activation, and R and T have the same meanings as in the Arrhenius equation. This equation is used for more complex reactions involving multiple transition states. It relates the rate constant to the free energy of activation required to overcome the energy barrier for the reaction to occur. The Eyring equation takes into account the entropy and enthalpy changes that occur during the reaction.

In some embodiments, the method solves for the parameters A1, E1, A2, E2 given the constraining data [AMINE], [CARB] and [CO2] and equations:

$$D [ AMINE ] / Dt = {A}_{1} {e}_{1}^{- E / RT} [ MEA ] - {A}_{2} {e}_{2}^{- E / RT} [ CARBM ] \\ [ AMINE ] + [ CARBM ] = [ AMINE ] 0$$

In the case of the Arrhenius form, the method includes solving for the Arrhenius constants for k (forward and reverse).

A variety of methods exist to solve for the 4 parameters given the arrays of data resulting from observation. In some embodiments, a statistical modeling software package that allows users to fit complex mathematical models to data may be used to determine one or more functions that fit the gathered data. In some instances, the Eyring form may also be computed with a model. In some instances, after running a test, the fully known set of equations and associated constants may comprise a complete model for the behavior of the amine that allows the prediction of the behavior of the amine solution under varying conditions in practical situations and plant models to estimate the optimal use parameters and cost of operations.

In some embodiments, method 500 continues with operation 520 of applying the results of operation 515 to modify a manufacturing process. For example, given a manufacturing process producing a gas with a particular concentration of waste CO2, the results of one or more rounds of testing may be used to select an amine solution and temperature for a process. For example, given the temperature swing parameters for a given manufacturing processs, the kinetics model can be used to simulate the performance of a given amine solution in the plant. Using these simulations to calculate the amount of CO2 captured for a given energy input, an amine with the highest performance may be selected for further testing.

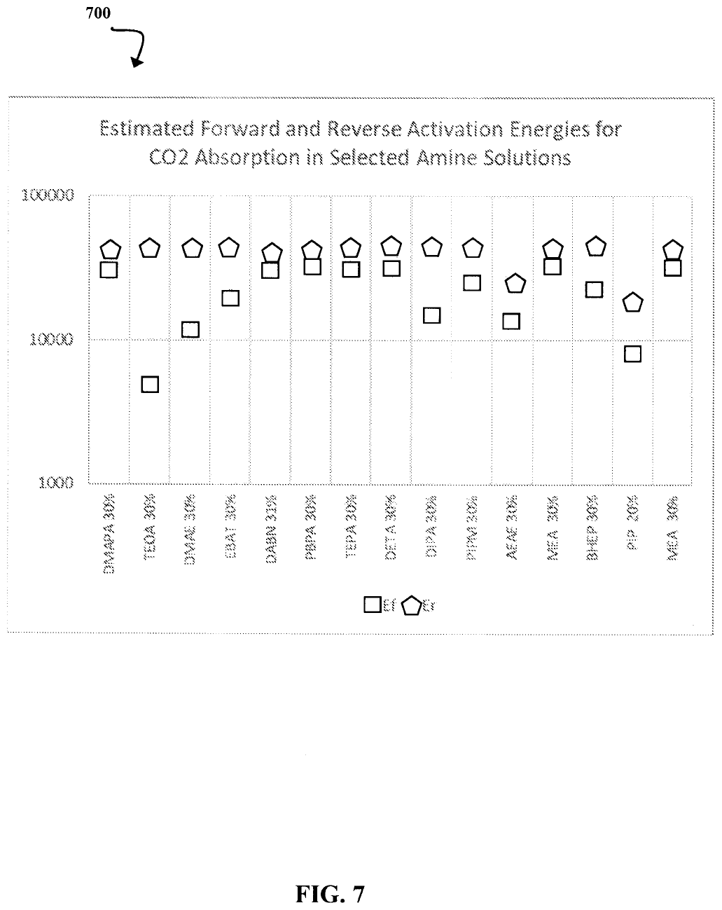

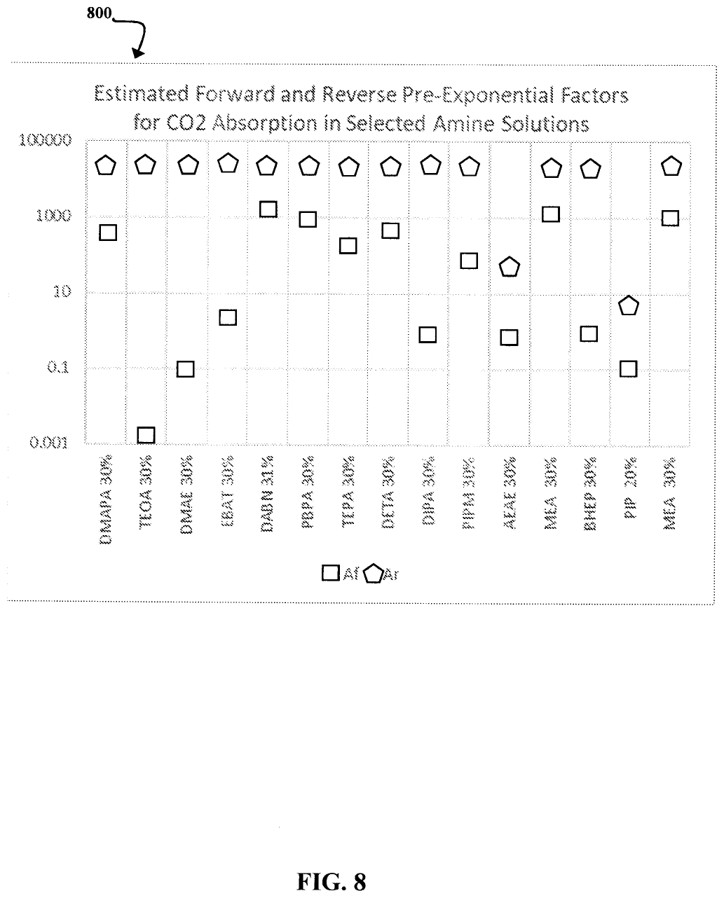

Using the method described above, multiple amine solutions were examined and the results of the examination were used to compute the corresponding first order reaction constants. These are shown in FIG. 7 and FIG. 8. FIG. 7 depicts graph 700 illustrating the estimated forward and reverse activation energies for CO2 absorption in various aqueous amine solutions. FIG. 8 depicts graph 800 illustrating the estimated forward and reverse pre-exponential factors (rate of the process) for CO2 absorption in various aqueous amine solutions. The 4 constants depicted for each amine solution, when plugged into the equations above, create a model for the performance of the amine that can then be applied to compute the performance of the amine usage in a given plant. The ability to model performance allows for fine tuning industrial processes without experimentation on the process itself. For example, tear down and setup on an industrial process to change CO2 capture methods in order to improve the methods may be cost prohibitive. However, by using an experimental process, as described herein, the CO2 capture methods for the industrial process may be improved without testing on the industrial setup itself. In some embodiments, Ef and Er refer to the forward and reverse reaction kinetics model constants, where E corresponds to energy. In some embodiments, Af and Ar refer to the forward and reverse reaction kinetics model constants where A is a pre-exponential constant.

In some instances, the amines along the X axis in FIGS. 7 and 8 are MEA: Monoethanolamine. MDEA: N-Methyldiethanolamine, DMAE: Dimethylaminoethanol, PIPM: Piperidinylpropylamine, DIPA: Diisopropanolamine, AEAE: 2-Aminoethyl-2-aminoethanol, TEOA: Triethanolamine, DETA: Diethylenetriamine, DMAE: Dimethylaminoethanol, BHEP: N-Butylpiperidinyl-4-amine, PIP: Piperazine, and MEA: Monoethanolamine.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

Computing environment 900 contains an example of an environment for the execution of at least some of the computer code involved in performing the inventive methods, such as and application for predicting the behavior of the amine solution under varying conditions to improve use parameters and cost of operations in example method 400. In addition to example method 400, computing environment 900 includes, for example, computer 901, wide area network (WAN) 902, end user device (EUD) 903, remote server 904, public cloud 905, and private cloud 906. In this embodiment, computer 901 includes processor set 910 (including processing circuitry 920 and cache 921), communication fabric 911, volatile memory 912, persistent storage 913 (including operating system 922 and example method 400, as identified above), peripheral device set 914 (including user interface (UI), device set 923, storage 924, and Internet of Things (IoT) sensor set 925), and network module 915. Remote server 904 includes remote database 930. Public cloud 905 includes gateway 940, cloud orchestration module 941, host physical machine set 942, virtual machine set 943, and container set 944.

COMPUTER 901 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 930. As is well understood in the art of computer technology, and depending upon the technology, performance of a computer-implemented method may be distributed among multiple computers and/or between multiple locations. On the other hand, in this presentation of computing environment 900, detailed discussion is focused on a single computer, specifically computer 901, to keep the presentation as simple as possible. Computer 901 may be located in a cloud, even though it is not shown in a cloud in FIG. 9. On the other hand, computer 901 is not required to be in a cloud except to any extent as may be affirmatively indicated.

PROCESSOR SET 910 includes one, or more, computer processors of any type now known or to be developed in the future. Processing circuitry 920 may be distributed over multiple packages, for example, multiple, coordinated integrated circuit chips. Processing circuitry 920 may implement multiple processor threads and/or multiple processor cores. Cache 921 is memory that is located in the processor chip package(s) and is typically used for data or code that should be available for rapid access by the threads or cores running on processor set 910. Cache memories are typically organized into multiple levels depending upon relative proximity to the processing circuitry. Alternatively, some, or all, of the cache for the processor set may be located “off chip.” In some computing environments, processor set 910 may be designed for working with qubits and performing quantum computing.

Computer readable program instructions are typically loaded onto computer 901 to cause a series of operational steps to be performed by processor set 910 of computer 901 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 921 and the other storage media discussed below. The program instructions, and associated data, are accessed by processor set 910 to control and direct performance of the inventive methods. In computing environment 900, at least some of the instructions for performing the inventive methods may be stored in example method 400 in persistent storage 913.

COMMUNICATION FABRIC 911 is the signal conduction paths that allow the various components of computer 901 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 912 is any type of volatile memory now known or to be developed in the future. Examples include dynamic type random access memory (RAM) or static type RAM. Typically, the volatile memory is characterized by random access, but this is not required unless affirmatively indicated. In computer 901, the volatile memory 912 is located in a single package and is internal to computer 901, but, alternatively or additionally, the volatile memory may be distributed over multiple packages and/or located externally with respect to computer 901.

PERSISTENT STORAGE 913 is any form of non-volatile storage for computers that is now known or to be developed in the future. The non-volatility of this storage means that the stored data is maintained regardless of whether power is being supplied to computer 901 and/or directly to persistent storage 913. Persistent storage 913 may be a read only memory (ROM), but typically at least a portion of the persistent storage allows writing of data, deletion of data and re-writing of data. Some familiar forms of persistent storage include magnetic disks and solid state storage devices. Operating system 922 may take several forms, such as various known proprietary operating systems or open source Portable Operating System Interface type operating systems that employ a kernel. The code included in example method 400 typically includes at least some of the computer code involved in performing the inventive methods.

PERIPHERAL DEVICE SET 914 includes the set of peripheral devices of computer 901. Data communication connections between the peripheral devices and the other components of computer 901 may be implemented in various ways, such as Bluetooth connections, Near-Field Communication (NFC) connections, connections made by cables (such as universal serial bus (USB) type cables), insertion type connections (for example, secure digital (SD) card), connections made though local area communication networks and even connections made through wide area networks such as the internet. In various embodiments, UI device set 923 may include components such as a display screen, speaker, microphone, wearable devices (such as goggles and smart watches), keyboard, mouse, printer, touchpad, game controllers, and haptic devices. Storage 924 is external storage, such as an external hard drive, or insertable storage, such as an SD card. Storage 924 may be persistent and/or volatile. In some embodiments, storage 924 may take the form of a quantum computing storage device for storing data in the form of qubits. In embodiments where computer 901 is required to have a large amount of storage (for example, where computer 901 locally stores and manages a large database) then this storage may be provided by peripheral storage devices designed for storing very large amounts of data, such as a storage area network (SAN) that is shared by multiple, geographically distributed computers. IoT sensor set 925 is made up of sensors that can be used in Internet of Things applications. For example, one sensor may be a thermometer and another sensor may be a motion detector.

NETWORK MODULE 915 is the collection of computer software, hardware, and firmware that allows computer 901 to communicate with other computers through WAN 902. Network module 915 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 915 are performed on the same physical hardware device. In other embodiments (for example, embodiments that utilize software-defined networking (SDN)), the control functions and the forwarding functions of network module 915 are performed on physically separate devices, such that the control functions manage several different network hardware devices. Computer readable program instructions for performing the inventive methods can typically be downloaded to computer 901 from an external computer or external storage device through a network adapter card or network interface included in network module 915.

WAN 902 is any wide area network (for example, the internet) capable of communicating computer data over non-local distances by any technology for communicating computer data, now known or to be developed in the future. In some embodiments, the WAN may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 903 is any computer system that is used and controlled by an end user (for example, a customer of an enterprise that operates computer 901), and may take any of the forms discussed above in connection with computer 901. EUD 903 typically receives helpful and useful data from the operations of computer 901. For example, in a hypothetical case where computer 901 is designed to provide a recommendation to an end user, this recommendation would typically be communicated from network module 915 of computer 901 through WAN 902 to EUD 903. In this way, EUD 903 can display, or otherwise present, the recommendation to an end user. In some embodiments, EUD 903 may be a client device, such as thin client, heavy client, mainframe computer, desktop computer and so on.

REMOTE SERVER 904 is any computer system that serves at least some data and/or functionality to computer 901. Remote server 904 may be controlled and used by the same entity that operates computer 901. Remote server 904 represents the machine(s) that collect and store helpful and useful data for use by other computers, such as computer 901. For example, in a hypothetical case where computer 901 is designed and programmed to provide a recommendation based on historical data, then this historical data may be provided to computer 901 from remote database 930 of remote server 904.

PUBLIC CLOUD 905 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 905 is performed by the computer hardware and/or software of cloud orchestration module 941. The computing resources provided by public cloud 905 are typically implemented by virtual computing environments that run on various computers making up the computers of host physical machine set 942, which is the universe of physical computers in and/or available to public cloud 905. The virtual computing environments (VCEs) typically take the form of virtual machines from virtual machine set 943 and/or containers from container set 944. It is understood that these VCEs may be stored as images and may be transferred among and between the various physical machine hosts, either as images or after instantiation of the VCE. Cloud orchestration module 941 manages the transfer and storage of images, deploys new instantiations of VCEs and manages active instantiations of VCE deployments. Gateway 940 is the collection of computer software, hardware, and firmware that allows public cloud 905 to communicate through WAN 902.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 906 is similar to public cloud 905, except that the computing resources are only available for use by a single enterprise. While private cloud 906 is depicted as being in communication with WAN 902, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 905 and private cloud 906 are both part of a larger hybrid cloud.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to explain the principles of the embodiments, the practical application or technical improvement over technologies found in the marketplace, or to enable others of ordinary skill in the art to understand the embodiments disclosed herein.

What is claimed is:

1. A system comprising:
a heat spreader with a sample holder configured to hold an amine solution;
a temperature sensor configured to measure a temperature of the amine solution;
a heating device and cooling device in the heat spreader;
a gas flow valve connected to a gas inlet tube,
wherein an end of the gas inlet tube is adapted to sit below a surface level of the amine solution; and
a CO2 (carbon dioxide) sensor connected to the gas outlet tube.

2. The system of claim 1, wherein the valve controls a flow rate of a gas flowing through the inlet tube.

3. The system of claim 1, wherein the heat spreader is configured to dissipate heat from the solution.

4. The system of claim 1, further comprising:
a second CO2 sensor connected to the gas inlet tube.

5. The system of claim 1, further comprising:
a pressure regulator connected to the gas inlet tube;
a filter connected to the gas inlet tube;
a mass flow controller connected to the gas inlet tube;
a flow meter connected to the gas inlet tube;
a pressure meter connected to the gas inlet tube; and
a temperature meter connected to the gas inlet tube.

6. The system of claim 1 further comprising:
a humidity sensor connected to the gas outlet tube; and
a temperature sensor connected to the gas outlet tube.

7. The system of claim 1, further comprising:
a thermal interface grease between the sample holder and the heat spreader.

8. The system of claim 1 further comprising:
a gas supply heat exchange duct to heat the heat spreader.

9. The system of claim 1 further comprising:
one or more heat sinks to remove heat from the system.

10. A method comprising:
placing an amine solution in a sample holder;
placing the sample holder in a receptacle in a heat spreader;
bringing a temperature of the amine solution to a desired temperature value;
valving a specified gas volume with a known CO2 concentration to the amine solution for a period of time sufficient to saturate the amine while monitoring a CO2 exhaust concentration using a CO2 sensor;
thermal cycling the amine solution by alternately heating and cooling the amine solution for fixed time periods and a number of cycles;
monitoring the CO2 exhaust concentration; and
calculating one or more carbon dioxide absorption kinetics parameters.

11. The method of claim 10, wherein the method further comprises:
monitoring a CO2 exhaust temperature, pressure, and relative humidity; and
presoaking the amine solution to saturate the amine with CO2.

12. The method of claim 11, wherein the thermal cycling is partially actuated by a thermo-electric module.

13. The method of claim 10, wherein the method further comprises:
selecting an amine for a manufacturing cycle using the calculated parameters.

14. The method of claim 10,
wherein the amine solution absorbs CO2 during a cooling phase of the thermal cycling, and
wherein the amine solution releases CO2 during a heating phase of the thermal cycling.

15. A method comprising:
determining, based on a result of a CO2 (carbon dioxide) absorption test, a capacity of an amine solution;
determining first order reaction kinetics parameters of the amine solution;
determining a change in amine concentration verses time; and
modifying a manufacturing process based on the first order reaction kinetics parameters of the amine solution and the change in amine concentration verses time.

16. The method of claim 15 further comprising:
determining a change in amine concentration vs. carbamate concentration; and
selecting, solution based on the determining a change in amine concentration verses time and the change in amine concentration vs. carbamate concentration, an amine.

17. The method of claim 15, wherein a net molar amount of CO2 absorbed is computed from an input flow volume, a CO2 concentration of a gas before passing through the amine solution and a CO2 concentration of a gas after passing through the amine solution.

18. The method of claim 17, wherein the capacity of the amine solution is computed by dividing moles of CO2 absorbed by moles of amine in the amine solution.

19. The method of claim 15, wherein the amine solution is agitated with an ultrasonic liner.

20. The method of claim 15, further comprising:
testing a viscosity of the amine solution.
